(19) United States
(12) Patent Application Publication
(10) Pub. No.: US 2024/0032618 A1
(43) Pub. Date: Feb. 1, 2024

(54) FLAT TEXTILE MATERIAL HAVING INTEGRATED POCKETS FOR RECEIVING ELECTRICAL AND/OR ELECTRONIC COMPONENTS FOR MEDICAL MEASUREMENT AND/OR CONTROL PURPOSES

(71) Applicant: KOB GmbH, Wolfstein (DE)

(72) Inventors: Sina Borczyk, Kaiserslautern (DE); Marcin Meyer, Wolfstein (DE)

(21) Appl. No.: 18/278,341

(22) PCT Filed: Dec. 21, 2021
(86) PCT No.: PCT/EP2021/086944

§ 371 Date: Aug. 22, 2023

(30) Foreign Application Priority Data
Feb. 24, 2021 (DE) 10 2021 104 437.5

(51) Int. Cl. A41D 1/00; D03D 11/02; D03D 15/56; D03D 1/00; A61B 5/11; A61B 5/00; A61N 1/36

(52) U.S. Cl. CPC A41D 1/005; D03D 11/02; D03D 15/56; D03D 1/0088; A61B 5/11; A61B 5/6831; A61N 1/36014; D10B 2401/18; D10B 2509/00; D10B 2501/00; A41D 2500/20

(57) ABSTRACT

The invention relates to a flat textile material (2) for applying to a human or animal body (4) and for receiving electrical and/or electronic components (6) for medical measurement and/or control purposes, comprising: a longitudinal direction (8); a transverse direction (10) running transversely to the longitudinal direction; a plane of extension (12); a thickness direction (13) orthogonal to the e plane of extension (12); at least one carrier portion (36), which extends in the longitudinal direction (8) and has a carrier layer (44); at N least one pocket portion (34), which extends in the longitudinal direction (8), adjoins the carrier portion (36) in the longitudinal direction (8) and has at least one pocket (22) for receiving the electrical and/or electronic components (6); wherein the at least one pocket (22) comprises a first pocket layer (38) nearer the body and a second pocket layer (40) farther from the body, which delimit a pocket space (42) therebetween; wherein the at least one pocket (22) has at least one pocket opening (30) accessible in the transverse direction (10); wherein the flat textile material (2) comprises warp threads (48) arranged in the longitudinal direction (8) and weft threads (50) arranged in the transverse direction (10), which form the carrier layer (44), the first pocket layer (38) and the second pocket layer (40); and wherein at least one flexible electrical conductor (46) extending in the longitudinal direction (8) and floating at least in parts is provided between the first pocket layer (38) and the second pocket layer (40) in the thickness direction (13).

The invention relates to a flat textile material, which is intended to be applied onto a human or animal body and to receive electrical and/or electronic components for medical measurement and/or control purposes.

US 2021/0118427 A1 discloses multilayer flat textile materials having cavities for receiving electronic control units, the electronic control units being embedded in a fabric and comprising contact points which, in order to transmit electrical signals, cooperate with electrically conductive threads provided at the cavity and incorporated into the fabric.

The object of the present invention is to provide a flat textile material of the type mentioned, which can be produced in a weaving process and which is suitable for securely receiving electrical and/or electronic components.

These are inter alia intended to mean microcontrollers, rechargeable batteries, batteries, sensors for recording body signals and/or movement signals, as well as actuators for acting on the body.

Preferably, the flat textile material should be suitable both for long-term use and for multiple use. In particular, the flat textile material could also be used to assist and monitor wound treatment and/or wound management, specifically as a wound compress or integrated into a wound compress, or a wound covering in the broadly interpreted sense.

Medical measurement purposes are intended in particular to mean the reception of (bio)physiological signals or potentials, for example the electrical resistance of the skin, the heartbeat or the movement of the body.

Medical control purposes are intended in particular to mean controlling the body and/or acting on the body, for example inducing a movement of the body, electrostimulation of the muscles and/or nerves, in the scope of diagnosis, therapy, rehabilitation and/or self-tracking.

In order to achieve this object, the invention provides a flat textile material having the features of claim 1.

A flat textile material is thus provided, which is configured as follows. The flat textile material comprises a longitudinal direction, a transverse direction running transversely to the longitudinal direction, an extent plane, a thickness direction orthogonal to the extent plane, at least one carrier section extending in the longitudinal direction and having a carrier layer, at least one pocket section, which extends in the longitudinal direction and follows on from the carrier section in the longitudinal direction, having at least one pocket for receiving the electrical and/or electronic components, wherein the at least one pocket comprises a first pocket layer facing toward the body and a second pocket layer facing away from the body, which delimit a pocket space between them, wherein the at least one pocket has at least one pocket opening that is accessible in the transverse direction, wherein the flat textile material comprises warp threads arranged in the longitudinal direction and weft threads arranged in the transverse direction, which form the carrier layer, the first pocket layer and the second pocket layer, wherein at least one flexible electrical conductor, which extends in the longitudinal direction and is arranged floating at least in sections, is provided between the first pocket layer and the second pocket layer in the thickness direction.

Producing the flat textile material in a weaving process allows continuous production of the flat textile material and of the pocket provided therein or thereon. Arranging the pocket in a pocket section of the flat textile material achieves controlled positioning of the electrical and/or electronic components that can be received therein. The pocket is accessible on two sides, or preferably on one side. Furthermore, by providing the flexible electrical conductors arranged floating between the pocket layers, improved and more reliable electrical connection is possible in the pocket section between the electrical and/or electronic component and the electrical conductor.

Because of the floating arrangement of the electrical conductor, larger-area connection can be achieved in comparison with the only pointwise connection known from the prior art between the component and the electrical conductor.

Furthermore, the first pocket layer facing toward the body protects the skin against unintended touching of the electrical and/or electronic component that can be received. The second pocket layer facing away from the body covers the electrical and/or electronic component at least partially and prevents unintended external touching and/or influencing of the electrical and/or electronic component received in the pocket.

Advantageously, the floatingly arranged electrical conductor may be formed from an electrically conductive filament, an electrically conductive thread or an electrically conductive copper wire. The electrical conductor is preferably weavable and elastically deformable, in particular flexible and/or extensible, along the longitudinal extent and/or orthogonally to the longitudinal extent. It is furthermore conceivable for the electrical conductor to comprise a plurality of filaments arranged mutually parallel in the longitudinal direction, in particular a fiber bundle, or a plurality of copper wires, in particular a stranded copper conductor formed from a plurality of copper wires.

When electrical conductors are referred to here, this means that such an electrical conductor comprises an electrically conductive material, in particular is coated with an electrically conductive material, or consists of an electrically conductive material. A conductor considered to be electrically conductive in this case has an electrical resistance of at most 2000 ohm/m, preferably at most 1000 ohm/m, preferentially at most 500 ohm/m. This means that a conductor portion with a length of 1 m has, in a resistance measurement that is conventional per se, an electrical resistance of at most 2000 ohms, or at most 1000 ohms, or at most 500 ohms, respectively.

Essentially, in a fabric, the warp and the weft threads are woven together, and for example in a plain weave, the weft threads are in each case alternately guided over a warp thread and under a warp thread and the warp threads are in each case guided alternately over a weft thread and under a weft thread. In the floating arrangement of the electrical conductor, in contrast to the plain weave, the electrical conductor is not arranged alternately over a weft thread and under a weft thread but instead the electrical conductor runs floating over a plurality, preferably a multiplicity, of weft threads.

A distance between successive connecting points will be referred to below as a floating distance. It has been found to be advantageous for the electrical conductor to have a floating distance in the pocket section of between 5 mm and 100 mm, in particular between 5 mm and 50 m, preferably between 5 mm and 30 mm, preferentially between 10 mm and 20 mm.

Because the conductor runs floating in the pocket section, the electrical conductor is readily accessible in the pocket, so that the electrical conductor may for example be separated by scissors, adapted in respect of insulation or coating, and/or provided with plugs or the like.

It is also conceivable for the electrical conductor to be arranged entirely floating in the pocket section, that is to say for it to have no connecting point with the pocket section there. The connecting points may be present in the carrier section and/or in the transition between the carrier section and the pocket section. An entirely floating arrangement of the electrical conductor in the carrier section has a high flexibility in respect of the connection to a component to be inserted, since the electrical conductor is mobile or displaceable because of the lack of connection in the pocket section.

In an alternating configuration of the flat textile material, a further carrier section follows on from the pocket section opposite the carrier section in the longitudinal direction, the pocket section being arranged between the carrier sections in the longitudinal direction.

In this embodiment, there is thus an arrangement: carrier section—pocket section—carrier section in the flat textile material in the longitudinal direction.

It may also be found to be advantageous for a further pocket section to follow on from the carrier section opposite the pocket section in the longitudinal direction, the carrier section being arranged between the pocket sections in the longitudinal direction. In this further embodiment, there is thus an arrangement: pocket section—carrier section—pocket section in the flat textile material in the longitudinal direction.

It may be found to be particularly advantageous for the flat textile material to be configured substantially endlessly in the longitudinal direction, with a carrier section and a pocket section respectively alternating. In this way, in particular, the advantages of continuous production in a weaving process can be fully exploited.

In respect of the reception of the electrical and/or electronic components, it has been found to be particularly advantageous for the first pocket layer and the second pocket layer to bear or lie loosely on one another at least in regions in the pocket section. Simple introduction of the component into the pocket is consequently possible.

In respect of continuous production, it is furthermore advantageous for the warp threads that extend in the longitudinal direction to be combined starting from the pocket section in the transition to the carrier section in order to form the carrier layer. If the flat textile material is configured respectively with a pocket section and a carrier section successively in the longitudinal direction, then multilayer pocket sections and single-layer carrier sections alternate. This has the advantage on the one hand that both the carrier layer and the pocket layer can be formed with the same warp threads, and on the other hand that a stable carrier layer can be formed in the carrier section.

It is furthermore advantageous for the at least one electrical conductor to be arranged on the carrier layer, and in particular partially enclosed by the carrier layer or held thereon in another way. In this way, the conductor is limited in movement in the transverse direction by the warp threads. For further fixing of the conductor, it is found to be advantageous for the conductor to be connected at least pointwise to the carrier layer. For this purpose, the electrical conductor may be incorporated into the carrier layer as part of the fabric, or the electrical conductor may be fixed to the carrier layer by a holding means.

In a further development of the flat material according to the invention, a plurality of electrical conductors, which run substantially parallel to one another in the longitudinal direction, are provided, the electrical conductors being spaced apart from one another in the transverse direction in such a way that there is no direct contact between two or more electrical conductors during intended use of the flat textile material. Consequently, different signals can be transmitted in and along the flat textile material. Furthermore, a secure connection can respectively be ensured between the conductors and different contact points of the received electrical and/or electronic component.

Furthermore, it is advantageous for the electrical conductor to be insulated at least in sections in the carrier section. Unintended touching or contacting of the electrical conductor by the adjacent skin or from the outside can thus be prevented.

In particular, it may furthermore be found to be advantageous for the at least one electrical conductor not to be insulated, or to be stripped, at least in sections in the pocket section, so that an electrical contact can be established between the at least one electrical conductor and the electrical and/or electronic component received.

The electrical conductor may be provided with exposed, uninsulated regions before weaving into the flat textile material. Alternatively, the electrical conductor may be introduced into the flat material while being fully insulated, and may then be stripped according to requirements in the pocket section. Contacting of the electrical conductor and of the received component preferably takes place on the side of the component facing away from the body, so that no elevated profile of the electrical conductor can be formed on the side facing toward the body.

According to one development of the flat material according to the invention, the at least one electrical conductor is separated in the pocket section so that this electrical conductor has two free conductor ends, in particular for contacting of the electrical and/or electronic component. Because of the floating of the electrical conductor in the pocket section, the conductor can readily be separated, for example with scissors, after introduction into the flat material.

It is then possible to provide the free conductor ends of the separated electrical conductor with at least one plug, so that electrical contacting with the received electrical and/or electronic component can be established by means of a plug connection. For this purpose, the plug and the component respectively provide terminals corresponding to one another. Consequently, easy attachment and removal of the component are possible by establishing and releasing the plug connection.

According to a further embodiment, it is found to be advantageous for the at least one pocket to be closed opposite the pocket opening in the transverse direction by combining the first pocket layer and the second pocket layer. In respect of the continuous production process, it is consequently possible to weave a flat textile material with pockets that have a pocket opening on one side in the transverse direction and are closed on the other side.

A side region follows on from the at least one pocket opposite the pocket opening in the transverse direction, the first pocket layer and the second pocket layer being connected to one another over an area in the side region, in particular with the weft threads of the first pocket layer and the second pocket layer that run in the transverse direction and the warp threads of the first pocket layer and the second pocket layer that run in the longitudinal direction being woven together in this side region. In respect of continuous producibility of the flat material, it is also conceivable for the transverse extent of the pocket section to correspond substantially to the transverse extent of the carrier section.

It is furthermore advantageous for a positioning means for positioning and/or retaining an electrical and/or electronic component that can be inserted into the pocket to be provided on or in the pocket. Accordingly, the component can be held securely in the pocket, and preferably fixed in a defined position, so that the contact points of the electrical and/or electronic component and the electrical conductor bear on one another or are assigned to one another. In particular, a hook on the component and an undercut in the pocket section, corresponding to the hook, may be provided as positioning means. A magnet on the component and a ferromagnetic material in the pocket section may also be envisioned.

According to another particularly advantageous embodiment, elastically extensible warp threads, which extend in the longitudinal direction, and/or elastically extensible weft threads, which extend in the transverse direction, are provided in the carrier section and/or in the pocket section. Consequently, a tight fit of the flat textile material on the body is ensured. Furthermore, the elastic threads in the pocket section may be configured in such a way that an introduced electrical and/or electronic component is enclosed retentively so that the component is prevented from falling out of the pocket. Furthermore, different elastic threads may be provided in the first pocket layer and the second pocket layer. In particular, it is conceivable for more elastic threads to be provided in the second pocket layer than in the first pocket layer. In this way, it is possible to ensure that the second pocket layer facing away from the body is more pliant than the first pocket layer facing toward the body when a component is introduced, so that the component is pushed away from the skin and therefore exerts less pressure on the patient's skin.

The elasticity of said elastic threads may be determined in a similar way to DIN EN ISO 13934-1. In this case, thread sections each having a length of 80 mm are used instead of the flat specimen mentioned in the standard. The elasticity of the thread is determined with the aid of the maximum tensile force extension of the thread used.

In this case, it is found to be advantageous for the maximum tensile force extension of the elastic threads to be at least 1.10 times, in particular at least 2.0 times and more particularly at least 5.0 times, in particular at least 10 times, in particular at least 20 times that of the other threads used. It is furthermore found to be advantageous for the maximum tensile force extension of the elastic threads to lie in a range of between 10% and 300%, particularly in a range of between 10% and 200%, advantageously in a range of between 10% and 100%, preferably in a range of between 10% and 20%.

The invention also relates to a holding or carrying device for an electrical and/or electronic component, which comprises a flat textile material according to the invention. The flat textile material is in this case preferably prefabricated as a collar, armband, headband, chest strap or kinesio tape.

It is furthermore conceivable for the flat material to be configured as a bandage or dressing, the endlessly produced flat material being cut to a defined length. Consequently, the flat material forms end regions with free ends in the longitudinal direction. The end regions of the flat material may be connected, in particular stitched, welded and/or adhesively bonded, to one another, so that an annular bandage or dressing is formed. Alternatively, carrier sections may be provided in the end region in the longitudinal direction, in which case hook-and-loop fasteners or adhesive may in particular be applied thereon.

By means of the hook-and-loop fasteners, dressings may be adapted expediently to the adjacent body part. By means of the adhesive, the flat material may be used as a plaster.

Protection is furthermore claimed for a device for medical measurement and/or control purposes, for use on a human or animal body, comprising a holding or carrying device according to the invention and an electrical and/or electronic component which is held and electrically contacted thereon, in particular a microcontroller, a battery, a rechargeable battery, a sensor for recording body signals and/or movement signals and/or a component for generating and/or emitting electrical signals onto the body. It is in this case conceivable for the component to be connected by means of a plug connection to the at least one electrical conductor. In the case of a cuboid component, the plug connection may be provided on the sides facing toward the carrier sections, on the side facing toward or away from the access opening, and/or on the sides of the component that face toward the pocket layers.

Further features, details and advantages of the invention may be found in the appended patent claims, the representation in the drawings and the following description of a preferred embodiment of the flat textile material according to the invention.

In the drawing:

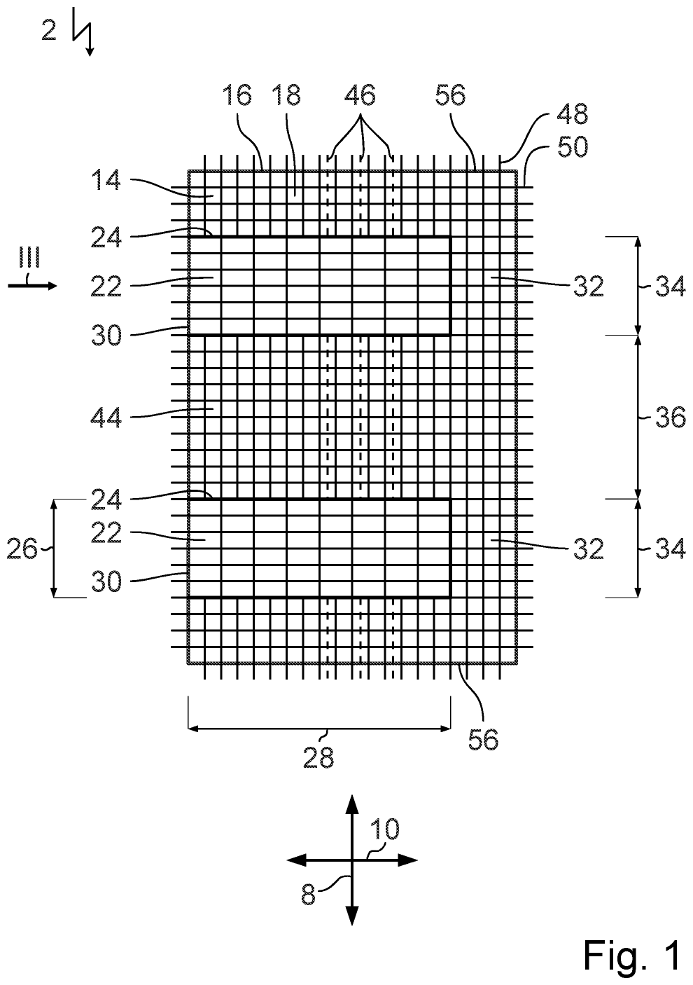

FIG. 1 shows a schematic plan view of a side of a flat textile material according to the invention facing toward the body;

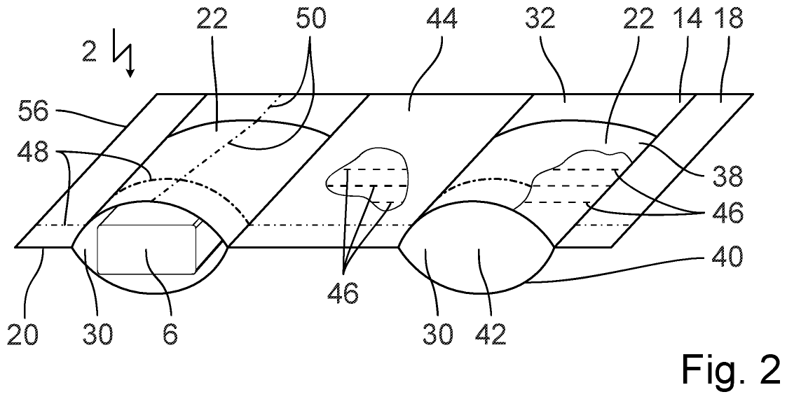

FIG. 2 shows a schematic three-dimensional representation of the flat textile material according to FIG. 1 with an inserted electronic component;

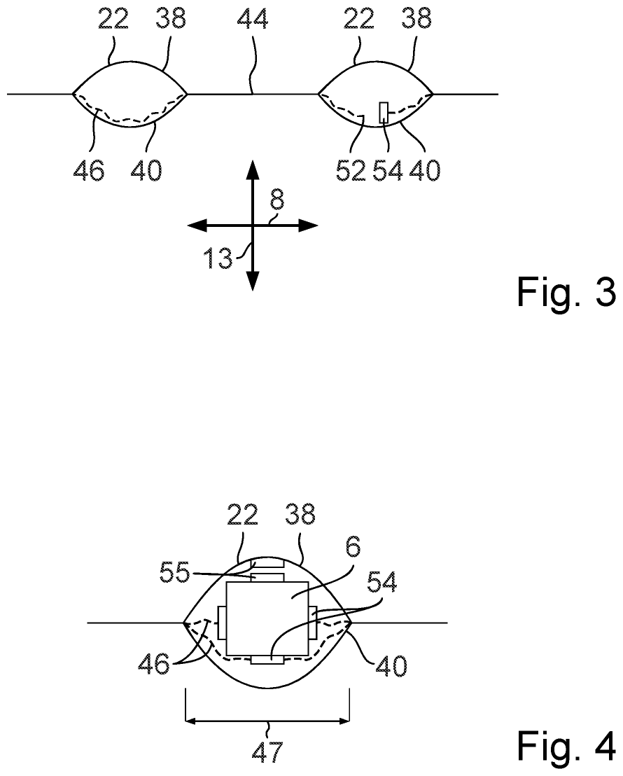

FIG. 3 shows a schematic side view of the flat material according to FIG. 1 as seen in the direction of the arrow III in FIG. 1;

FIG. 4 shows a schematic side view of the flat material according to FIGS. 1 and 2 with an electronic component inserted into a pocket;

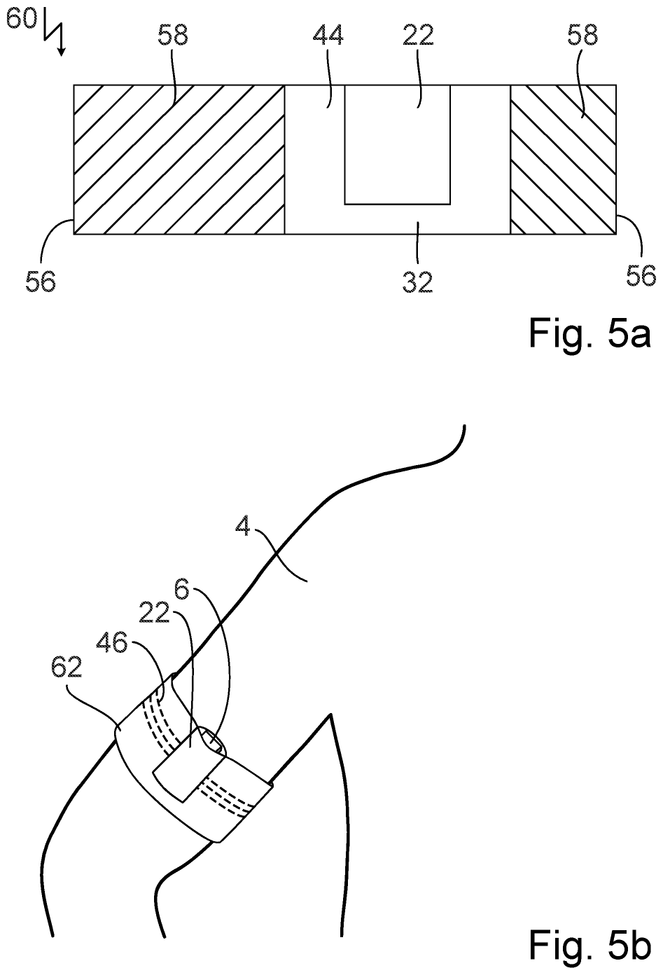

FIG. 5a shows a schematic representation of a flat textile material according to the invention, configured as a bandage, with holding means; and

FIG. 5b shows a schematic representation of a bandage with an electronic component received therein in a position of use on a person's upper arm.

The figures show a flat textile material 2 according to the invention and illustrate its structure, sometimes very schematically. The flat material 2 is intended to be applied onto a human or animal body 4 and to receive at least one electrical and/or electronic component 6. The flat textile material 2 extends in a longitudinal direction 8, in a transverse direction 10 and in an extent plane 12, which corresponds to the plane of the drawing of FIG. 1. A thickness direction 13, which can be seen in FIG. 3 and runs orthogonally to the extent plane 12, is furthermore introduced.

FIG. 1 shows a plan view of a side 14, facing toward the body, of a section 18, indicated by a rectangular box 16, of the flat textile material 2. FIG. 2 shows a three-dimensional representation of the flat textile material 2, the side 14 facing toward the body being oriented upward and the side 20—not visible—facing away from the body being oriented downward.

In order to receive electrical and/or electronic components 6, the section 18 of the flat textile material 2 comprises two pockets 22, in the case represented here configured rectangular by way of example, which are indicated in FIG. 1 by a box 24. The pockets 22 extend in the longitudinal direction 8 over a pocket length 26 and in the transverse direction 10 over a pocket width 28. The pockets 22 are accessible in the transverse direction 10 through a pocket opening 30 provided on a lateral edge of the section 18. Opposite the pocket opening 30 in the transverse direction 10, a side region 32 of the section 18 follows on from the pocket 22. The side region 32 extends in the longitudinal direction 8 over the pocket length 26 and in the transverse direction 10 between the pocket 22 and a lateral edge of the section 18.

The region of the respective pocket 22 respectively forms, together with the adjacent side region 32, a pocket section 34 of the flat textile material 2, or of the section 18. A carrier section 36 is provided, or arranged, between the two pocket sections 34 in the longitudinal direction 8.

As will be explained in more detail below, the flat textile material 2 according to the invention may be produced endlessly in the longitudinal direction 8 so that, from an endlessly produced strip or band of the flat textile material 2 according to the invention, the sections 18 of the flat material 2 that are represented in the figures may be obtained as longitudinal sections of this endless flat material 2. Thus, it is conceivable for a section 18 to be able to have one or more pockets 22, between which a carrier section 36 is respectively arranged—as represented—in the longitudinal direction 8.

The section 18 of the flat textile material 2 is likewise shown in FIG. 2, in which it can be seen that the flat textile material 2 comprises a first pocket layer 38 facing toward the body and a second pocket layer 40 facing away from the body in the respective pocket section 34. The first pocket layer 38 facing toward the body and the second pocket layer 40 facing away from the body may, in the preferentially represented case, in particular be applied loosely against one another and/or widened in such a way that a pocket space 42 is delimited by the first pocket layer 38 and the second pocket layer 40. The electrical and/or electronic component 6 can be received in this pocket space 42.

With the aid of FIG. 2 to 4, it may be seen that the first pocket layer 38 facing toward the body and the second pocket layer 40 facing away from the body are combined to form a textile carrier layer 44 provided in the carrier section 36. The carrier layer 44 extends in the transverse direction 10 between the lateral edges of the section 18 of the flat material 2, that is to say over its entire width.

In the longitudinal direction 8, the carrier layer 44 extends between the two pockets 22.

Preferably, besides the function of receiving the electrical and/or electronic component 6, the flat textile material 2 fulfills the function of electrical connection of the received component 6 to further electrical and/or electronic components, which lie outside this pocket 22.

These may for example be arranged in further pockets 22 of the same flat textile material 2, or for example may be configured and arranged as an electrode in the form of an electrode section on a further functional section of the flat textile material 2.

For connection, or contacting, of the electrical and/or electronic component 6, in this embodiment by way of example three electrical conductors 46 are provided, which are introduced into the flat textile material 2. In FIG. 1 to 5, the electrical conductors 46 are represented as dashed lines. The electrical conductors 46 are used to transmit signals away from the body, toward the body and/or between two electrical and/or electronic components 6, each electrical conductor 46 being able to transmit a different signal. The electrical conductors 46 are arranged running in the longitudinal direction 8 and parallel to one another. In the carrier section 36, the electrical conductors 46 are connected to the carrier layer 44 in such a way that unintended slipping of the electrical conductors 46 in the longitudinal direction 8 and/or in the transverse direction 10 is prevented. Furthermore, the electrical conductors 46 are incorporated into the carrier layer 44 in such a way that the electrical conductors 46 can be prevented from touching one another during intended use. In the pocket section 34, furthermore, the electrical conductors 46 are not attached to the first pocket layer 38 and not attached to the second pocket layer 40.

Consequently, the electrical conductors 46 lie substantially freely in the pocket 22 between the first pocket layer 38 and the second pocket layer 40, in which case they may bear loosely from the inside on the first pocket layer 38 and/or on the second pocket layer 40.

The formation of the layers, the pockets, the regions and the sections of the flat textile material according to the invention will be explained below.

The first pocket layer 38, the second pocket layer 40 and the carrier layer 44 comprise warp threads 48 extended in the longitudinal direction 8 and weft threads 50 extended in the transverse direction 10, which in particular are configured as electrically nonconductive threads.

The courses of a single warp thread 48 and of a single weft thread 50 are schematically indicated in FIG. 2. The warp thread 48 and the weft thread 50 are lowered from the neutral position and woven together in order to form the second pocket layer 40 in the pocket section 34. Similarly, further warp threads 48 and weft threads 50 are raised from the neutral position and woven together in order to form the first pocket layer 38. At the transition to the pocket section, the warp threads 48 and the weft threads 50 are respectively raised or lowered back into the neutral position. Thus, the warp threads 48 that extend in the longitudinal direction 8 and form the first pocket layer 38, and the warp threads 48 that extend in the longitudinal direction 8 and form the second pocket layer 40 are combined in order to form the woven carrier layer 44 in the carrier section 36. Correspondingly, a stable carrier layer 44 is formed from the warp threads 48 and the weft threads 50 in the neutral position.

The formation of the side region 32 is carried out correspondingly. The weft threads 50, which form in each case the first pocket layer 38 and the second pocket layer 40, are respectively raised or lowered in the region of the pocket 22. At the transition from the pocket 22 to the side region 32, the respective weft threads 50 are lowered or raised back into the neutral position and woven with further warp threads 48 so that a single textile layer, which is comparable to the carrier layer 44, can be formed in the side region 32. Alternatively, the two pocket layers 38, 40 could also be connected to one another there subsequently in any desired way.

The course of the electrical conductor 46 may be seen particularly in FIGS. 2 and 3. The electrical conductors 46 are covered almost fully in the carrier section 36 by the warp threads 48 and the weft threads 50 in the carrier section 36. In the pocket section 34, the electrical conductors 46 are for example arranged entirely floating.

Thus, the electrical conductors 46 are not attached to the first pocket layer 38 and not attached to the second pocket layer 40 in the pocket section 34. The electrical conductors 46 are freely mobile in the pocket 22 within the scope of their play and elasticity, the electrical conductors 46 respectively having two connecting points in the transition between the pocket section 34 and the carrier section 36. Consequently, the floating distance 47 of the electrical conductors 46 in the pocket section 34 corresponds approximately to the pocket length 26. It is by way of example conceivable for the electrical conductors 46 to be arranged floating on the first pocket layer 38 and/or on the second pocket layer 40 with a floating distance 47 of between 5 mm and 30 mm. With an exemplary pocket length 26 of 30 mm and a floating distance 47 of 5 mm, the electrical conductors 46 are attached in the transitions between the pocket section 34 and the carrier section 36 and at five further connecting points. In this case, six floating regions of the electrical conductors 46 are formed in the pocket section 34. It is furthermore possible, in the case of a plurality of electrical conductors 46, for the individual electrical conductors 46 to be arranged differently in the pocket section 34 and/or in the carrier section 36, in particular for them to be arranged floating differently in the pocket section 34. The electrical conductors 46 may each have a floating distance in the pocket section of preferably between 5 mm and 100 mm, in particular between 5 mm and 50 mm, preferably between 5 mm and 30 mm, preferentially between 10 mm and 20 mm.

The floating arrangement of the electrical conductors 46 is indicated in FIG. 3 as an undulating dashed line. In the left pocket 22, the electrical conductor 46 is continuous, in which case for example an electrical and/or electronic component 6 having open contact points may be inserted into the pocket 22 so that a bearing connection may be established between the component 6 and the electrical conductors 46 by the alignment of the component 6 in the pocket 22. Accordingly, it is expedient for the electrical conductors 46 to be electrically contactable in the pocket section 34. The electrical conductors 46 may be introduced into the flat textile material 2 with insulation. The electrical conductors 46 then need to be stripped in the pocket section 34. It is furthermore conceivable for the electrical conductors 46 not to have any insulation in sections and for the uninsulated regions to be aligned with the pocket sections 34 when the electrical conductors 46 are introduced into the flat material 2. In the right pocket 22, the electrical conductor 46 has been separated by means of scissors. Consequently, the electrical conductor 46 has two free conductor ends 52. Depending on the type of conductor, the free conductor end 52 may be connected to a component 6 to be inserted. The left free conductor end 52 may for example be joined, in particular adhesively bonded or soldered, onto a contact point of a component 6 to be inserted. Provided at the right free conductor end 52, there is a plug 54 with which a plug connection can be established with a component 6 to be inserted. The electrical and/or electronic component 6 accordingly has a socket corresponding to the plug 54.

FIG. 4 shows a pocket 22 of a flat material 2 according to the invention. An electronic component 6, which comprises four sockets for plugs 54, is inserted into the pocket 22. The flat material 2 in this case comprises two electrical conductors 46, which are equipped with plugs 54 at their free conductor ends 52. The plugs 54 are plugged into the electronic component 6 in the longitudinal direction 8, or in the thickness direction 13. For simplified and better alignment of the electrical and/or electronic component, positioning means 55 are provided on the component 6 and the pocket 22, which, when the component 6 is inserted, cooperate in such a way that the component 6 and the pocket 22 engage and/or the user feels a resistance. For example, magnets, locking hooks with undercuts and/or adhesives may be envisioned as positioning means 55.

FIGS. 5a and 5b show by way of example the flat textile material 2 according to the invention, which is designed as a bandage 60 to be arranged on an arm. The bandage 60 comprises a pocket 22 and two carrier layers 44, which adjoin the pocket 22 in the longitudinal direction 8. The bandage 60 and the electronic component 6 received in the pocket 22 form a device 62 for medical measurement and/or control purposes. In the case of the bandage 60, the carrier layers 44 respectively end in the longitudinal direction 8 in a free carrier end 56. Holding means 58 for establishing the adhesion, or fixing, on the arm may respectively be provided on the free carrier ends 56 of the carrier layers 44. For example, these holding means 58 may be configured as adhesive points so that the flat material 2 can be adhesively bonded onto the skin as a plaster. As an alternative, FIG. 5b shows a bandage 60 which has a hook-and-loop fastener on the free carrier ends 56 of the carrier layers 44. Consequently, the free carrier ends 56 may be placed above one another so that the flat material 2 can be fixed individually on the arm. It is furthermore conceivable for the free carrier ends 56 to be stitched to one another, so that a bandage 60 having a defined circumference can be produced from the flat material 2. In this case, it is particularly advantageous for the flat material 2 to have elastic threads 48, 50 so that an elastically extensible bandage 60, which fits tightly to the skin, can be produced. The elastic threads 48, 50 may also advantageously be used in the other embodiments.

The elastic threads 48, 50 may thus also be advantageous when they are provided in the region of the pocket layer 38, 40, so that a component 6 received therein can be enclosed by one or both pocket layers 38, 40 and thus cannot fall out of the pocket 22.

What is claimed is:

1. A flat textile material (2), which is intended to be applied onto a human or animal body (4) and to receive an electrical and/or electronic component (6) for medical measurement and/or control purposes, the flat textile material (2) comprising:
a longitudinal direction (8);
a transverse direction (10) running transversely to the longitudinal direction (8);
an extent plane (12);
a thickness direction (13) orthogonal to the extent plane (12);
at least one carrier section (36) extending in the longitudinal direction (8) and having a carrier layer (44); and
at least one pocket section (34), which extends in the longitudinal direction (8) and follows on from the carrier section (36) in the longitudinal direction (8), having at least one pocket (22) for receiving the electrical and/or electronic component (6),
wherein the at least one pocket (22) comprises a first pocket layer (38) facing toward the body (4), and a second pocket layer (40) facing away from the body (4), which delimit a pocket space (42) between them,
wherein the at least one pocket (22) has at least one pocket opening (30) that is accessible in the transverse direction (10),
wherein the flat textile material (2) comprises warp threads (48) arranged in the longitudinal direction (8) and weft threads (50) arranged in the transverse direction (10), which form the carrier layer (44), the first pocket layer (38), and the second pocket layer (40), and
wherein at least one flexible electrical conductor (46), which extends in the longitudinal direction (8) and is arranged floating at least in sections, is provided between the first pocket layer (38) and the second pocket layer (40) in the thickness direction (13).

2. The flat textile material (2) of claim 1, wherein the floatingly arranged electrical conductor (46) has a floating distance (47) of at least 5 mm and/or at most 100 mm.

3. The flat textile material (2) of claim 1, further comprising another carrier section (36) that follows on from the pocket section (34) opposite the carrier section (36) in the longitudinal direction (8), the pocket section (34) being arranged between the carrier sections (36) in the longitudinal direction (8).

4. The flat textile material (2) of claim 1 further comprising another pocket section (34) that follows on from the carrier section (36) opposite the pocket section (34) in the longitudinal direction (8), the carrier section (36) being arranged between the pocket sections (34) in the longitudinal direction (8).

5. The flat textile material (2) of claim 1, wherein the flat textile material (2) is configured endlessly in the longitudinal direction (8), with a carrier section (36) and a pocket section (34) respectively alternating.

6. The flat textile material (2) of claim 1, wherein the first pocket layer (38) and the second pocket layer (40) bear or lie loosely on one another at least in regions in the pocket section (34).

7. The flat textile material (2) of claim 1, wherein the warp threads (48) that extend in the longitudinal direction (8) are combined starting from the pocket section (34) in the transition to the carrier section (36) in order to form the carrier layer (44).

8. The flat textile material (2) of claim 1, wherein the at least one electrical conductor (46) is arranged on the carrier layer (44).

9. The flat textile material (2) of claim 1, wherein the at least one electrical conductor (46) comprises a plurality of electrical conductors (46), which run substantially parallel to one another in the longitudinal direction (8), the electrical conductors (46) being spaced apart from one another in the transverse direction (10) in such a way that there is no direct contact between two or more electrical conductors (46) during intended use of the flat textile material (2).

10. The flat textile material (2) claim 1, wherein the at least one electrical conductor (46) is insulated at least in sections in the carrier section (36).

11. The flat textile material (2) of claim 1, wherein the at least one electrical conductor (46) is not insulated, or is stripped, at least in sections in the pocket section (34), so that an electrical contact can be established between the at least one electrical conductor (46) and the electrical and/or electronic component (6).

12. The flat textile material (2) of claim 1, wherein the at least one electrical conductor (46) is separated in the pocket section (34) so that the electrical conductor (46) has two free conductor ends (52).

13. The flat textile material (2) of claim 12, further comprising a plug (54) that is provided on at least one free conductor end (52) of the separated electrical conductor (46), so that a plug connection can be established with the electrical and/or electronic component (6).

14. The flat textile material (2) of claim 1, wherein the at least one pocket (22) is closed opposite the pocket opening (30) in the transverse direction (10) by combining the first pocket layer (38) and the second pocket layer (40).

15. The flat textile material (2) of claim 1, further comprising a side region (32) that follows on from the at least one pocket (22) opposite the pocket opening (30) in the transverse direction (10), the first pocket layer (38) and the second pocket layer (40) being connected to one another over an area in the side region (32).

16. The flat textile material (2) of claim 1, further comprising a positioning means (55) on or in the pocket (22) for positioning and/or retaining the electrical and/or electronic component (6) that can be inserted into the pocket (22).

17. The flat textile material (2) of claim 1, wherein the warp threads (48) are elastically extensible warp threads (48), which extend in the longitudinal direction (8), and/or the weft threads (50) are elastically extensible weft threads (50), which extend in the transverse direction (10), and wherein the elastically extensible warp threads (48) and/or the elastically extensible weft threads (50) are provided in the carrier section (36) and/or in the pocket section (34).

18. A holding or carrying device (60) for an electrical and/or electronic component (6), the holding or carrying device (60) comprising:
a flat textile material (2), which is intended to be applied onto a human or animal body (4) and to receive the electrical and/or electronic component (6) for medical measurement and/or control purposes, the flat textile material (2) comprising:
a longitudinal direction (8);
a transverse direction (10) running transversely to the longitudinal direction (8);
an extent plane (12);
a thickness direction (13) orthogonal to the extent plane (12);
at least one carrier section (36) extending in the longitudinal direction (8) and having a carrier layer (44); and
at least one pocket section (34), which extends in the longitudinal direction (8) and follows on from the carrier section (36) in the longitudinal direction (8), having at least one pocket (22) for receiving the electrical and/or electronic components (6),
wherein the at least one pocket (22) comprises a first pocket layer (38) facing toward the body (4), and a second pocket layer (40) facing away from the body (4), which delimit a pocket space (42) between them,
wherein the at least one pocket (22) has at least one pocket opening (30) that is accessible in the transverse direction (10),
wherein the flat textile material (2) comprises warp threads (48) arranged in the longitudinal direction (8) and weft threads (50) arranged in the transverse direction (10), which form the carrier layer (44), the first pocket layer (38), and the second pocket layer (40), and
wherein at least one flexible electrical conductor (46), which extends in the longitudinal direction (8) and is arranged floating at least in sections, is provided between the first pocket layer (38) and the second pocket layer (40) in the thickness direction (13).

19. A device (62) for medical measurement and/or control purposes, for use on a human or animal body (4), the device (62) comprising:
a holding or carrying device (60); and
an electrical and/or electronic component (6) which is held and electrically contacted thereon, in particular a microcontroller, a battery, a rechargeable battery and/or a sensor for recording body signals and/or movement signals or a component for generating and/or emitting electrical signals onto the body (4),
the holding or carrying device (60) comprising:
a flat textile material (2), which is intended to be applied onto the human or animal body (4) and to receive the electrical and/or electronic component (6) for medical measurement and/or control purposes, the flat textile material (2) comprising:
a longitudinal direction (8);
a transverse direction (10) running transversely to the longitudinal direction (8);
an extent plane (12);
a thickness direction (13) orthogonal to the extent plane (12);
at least one carrier section (36) extending in the longitudinal direction (8) and having a carrier layer (44); and
at least one pocket section (34), which extends in the longitudinal direction (8) and follows on from the carrier section (36) in the longitudinal direction (8), having at least one pocket (22) for receiving the electrical and/or electronic components (6),
wherein the at least one pocket (22) comprises a first pocket layer (38) facing toward the body (4), and a second pocket layer (40) facing away from the body (4), which delimit a pocket space (42) between them,
wherein the at least one pocket (22) has at least one pocket opening (30) that is accessible in the transverse direction (10),
wherein the flat textile material (2) comprises warp threads (48) arranged in the longitudinal direction (8) and weft threads (50) arranged in the transverse direction (10), which form the carrier layer (44), the first pocket layer (38), and the second pocket layer (40), and
wherein at least one flexible electrical conductor (46), which extends in the longitudinal direction (8) and is arranged floating at least in sections, is provided between the first pocket layer (38) and the second pocket layer (40) in the thickness direction (13).

20. The flat textile material (2) of claim 15, wherein the first pocket layer (38) and the second pocket layer (40) are connected to one another over an area in the side region (32), with the weft threads (50) that run in the transverse direction (10) and the warp threads (48) that run in the longitudinal direction (8) being woven together in this side region (32).
